(19) United States
(12) Patent Application Publication
(10) Pub. No.: US 2011/0040264 A1
(43) Pub. Date: Feb. 17, 2011

(54) Medicated footwear

(75) Inventor: Gelcys Basulto, Studio City, CA (US)

Correspondence Address: Gelcys Basulto, 4332 Gentry Ave., #2, Studio City, CA, 91604, US

(73) Assignee: Cuban Element, Inc.

(21) Appl. No.: 12/157,305

(22) Filed: Jun. 10, 2008

(51) Int. Cl. A61M 35/00; A61F 7/08

(52) U.S. Cl. 604/293; 607/108

(57) ABSTRACT

A unique, previously unforeseen medicated footwear that is elastic in nature to fit to the size of the foot, and all sizes of feet. The consumer is any adult that wears shoes or sandals, and the device can be worn in bare feet while sitting down. The medication will both warm and relax the foot, and the varied uses imply relaxation, medication, heating (whether inside or outside), and the device will also protect the life of the shoe or sandal. The individual places the covering on his or her feet, in a sitting or walking position, and after use, disposes of the devices themselves for replacement. The products will be available in most pharmacies, and sold where heating medications and pads are offered to the public.

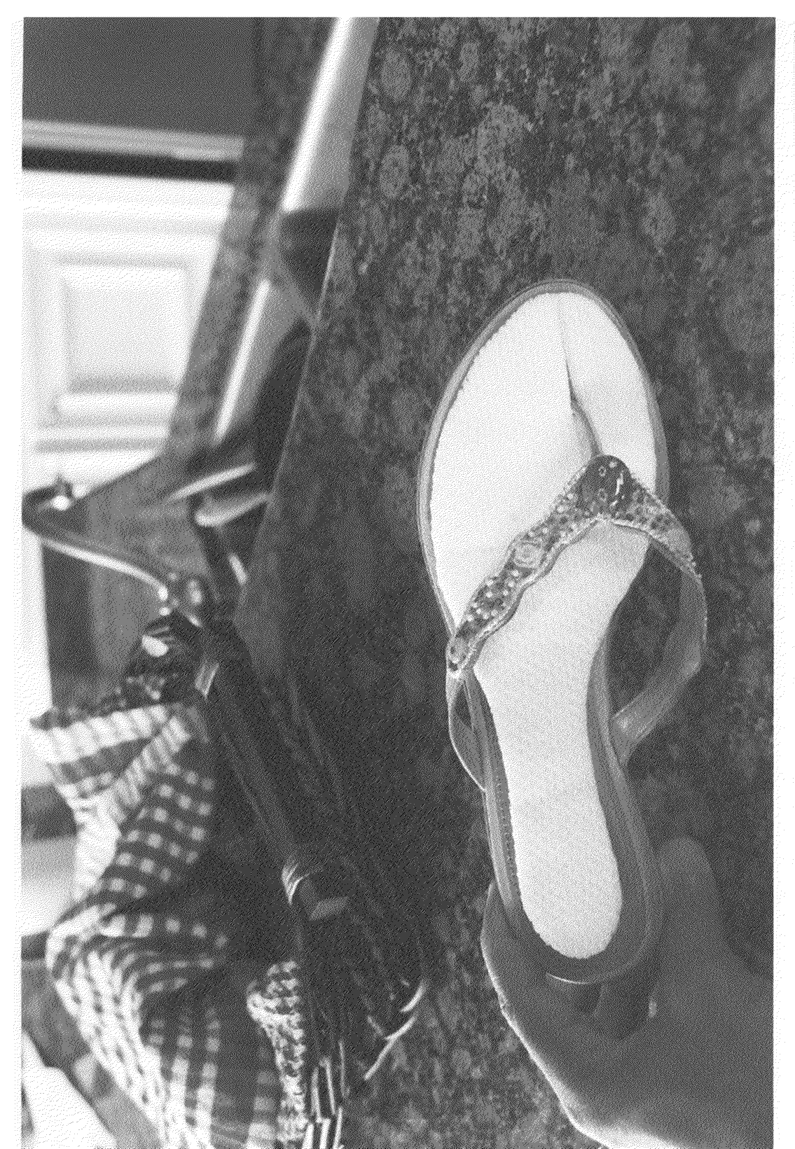

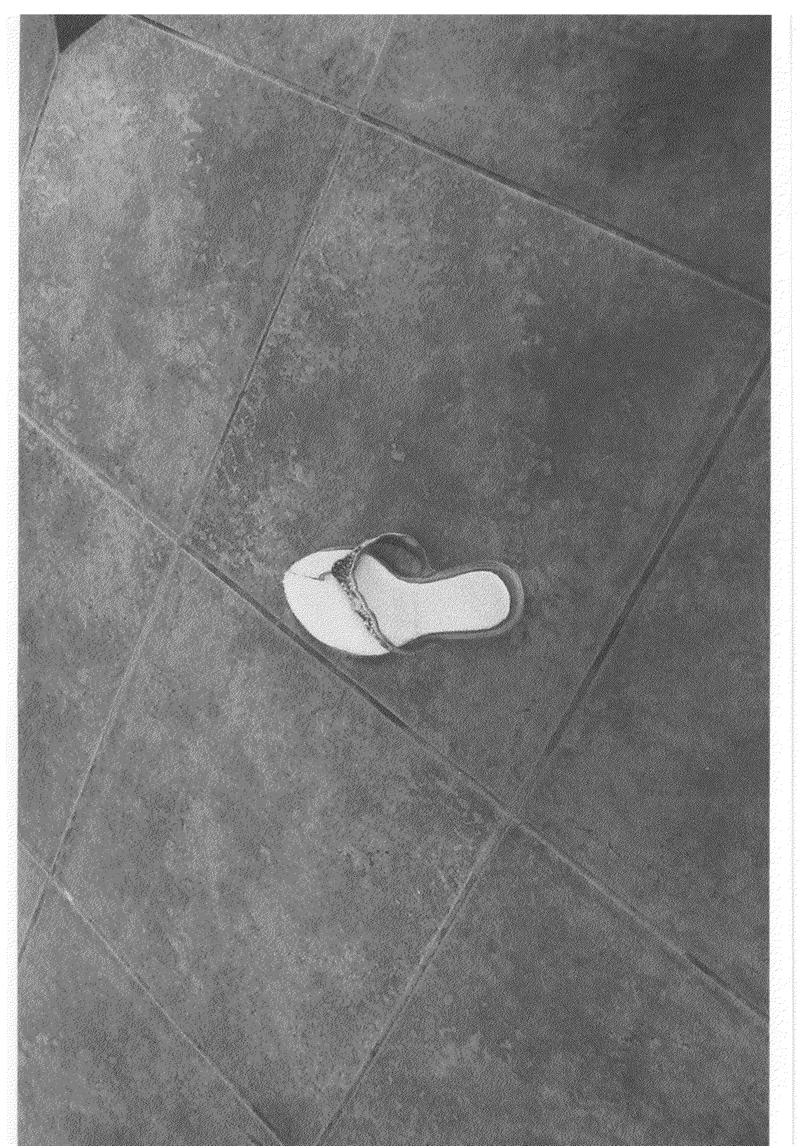

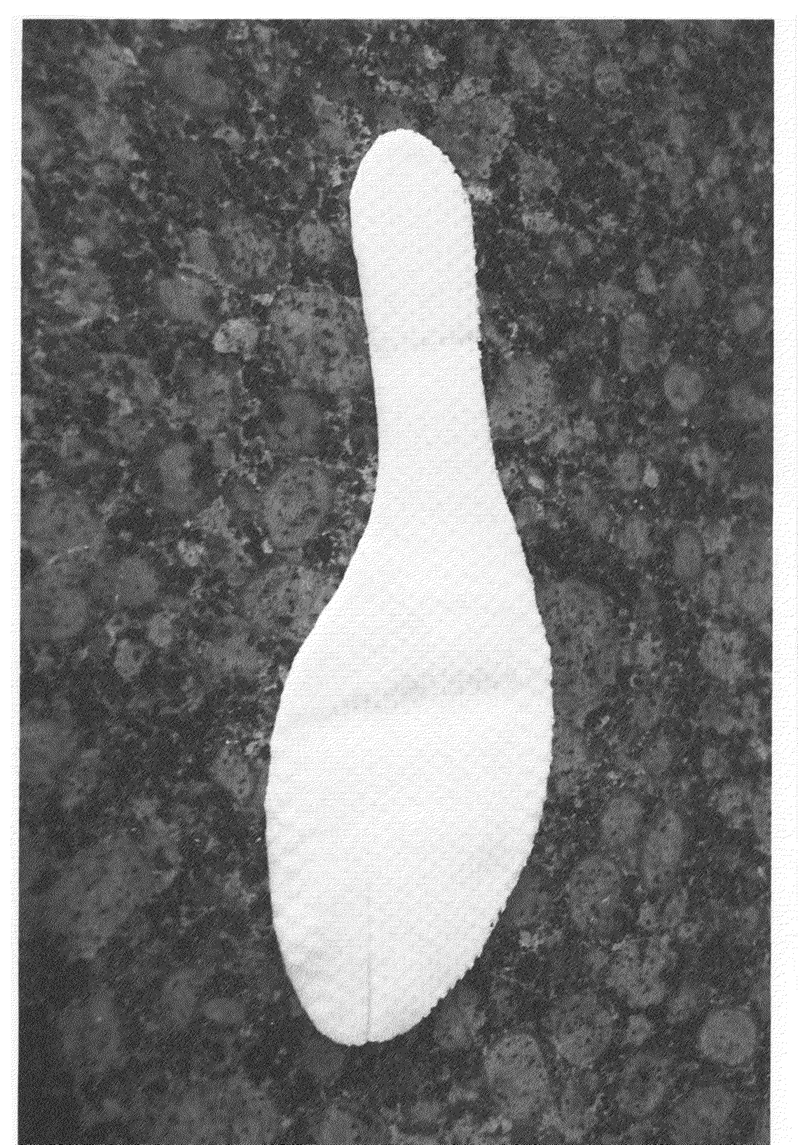

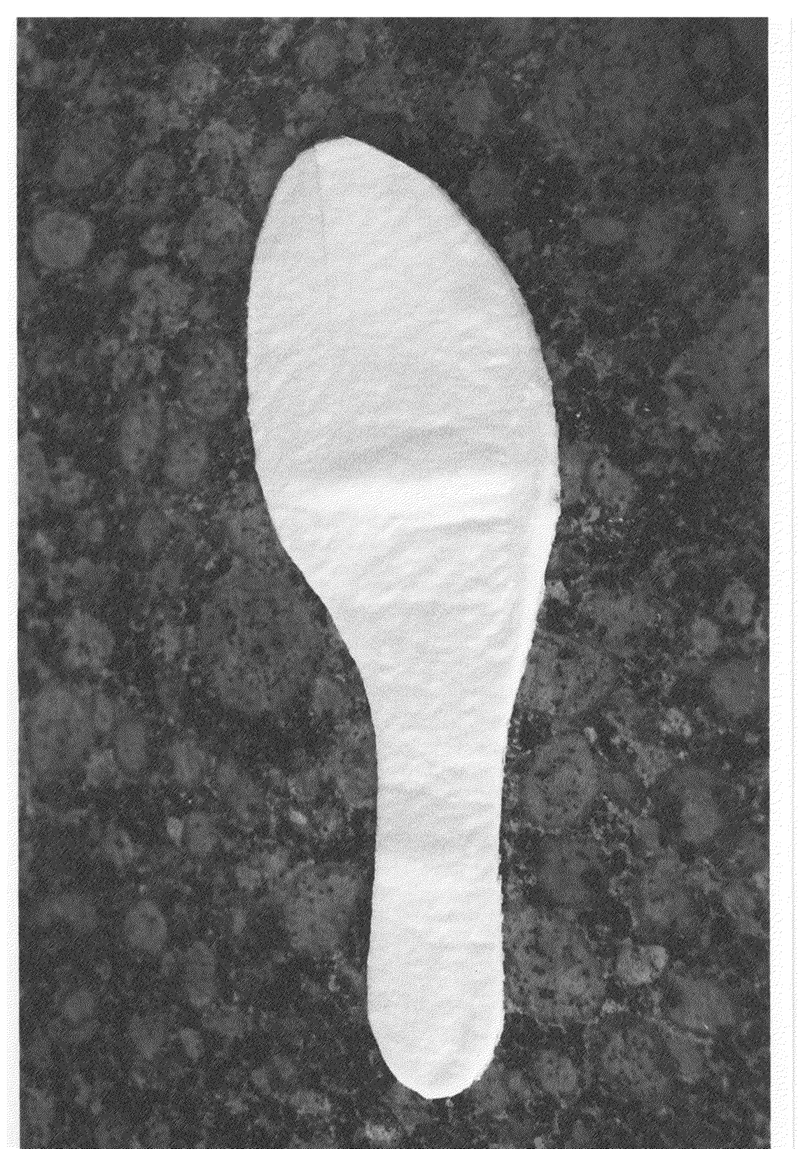

### OBJECT OF THE INVENTION

This invention is meant to solve the problem with foot attachments that are solely designed to be married to the shoe or sandal itself. The device in question will be disposable, attached to the foot itself, be able to be worn with or without the shoe or sandal, be low cost, and be medicated. The actual material of the device has never been used before, and the device itself will be medicated to provide foot care and heat, whether worn with or without the shoe. It will provide foot care and medication for the athlete, or simply a relaxation and foot care device for a party at rest. It is unique in its multiple application and attachment to the foot itself in medicated form.

### BACKGROUND OF THE INVENTION

1. The Invention Itself

This invention relates to medicated footwear, unlike mules, scuffs and sandals. It is attached to the foot or feet, it exists for the benefit of the foot, and is disposable after one usage.

2. Description of the Prior Art

There are mules and scuffs for common use in hotels, restaurants, hospitals and meeting places, so that anyone may use. Since the mules and scuffs provided in such public facilities are used by all sorts and conditions of people, it is necessary to maintain good hygiene of the footwear.

There has been proposed an idea of keeping the footwear clean, in which a cover sheet is removably attached onto an insole by use of a flat fastener, so that the cover sheet can be replaced when soiled (Japenese Utility Model Public Disclosures Nos. SHO 57-186504(A); SHO 59-243504(A); and HEI 1-169103 (A)).

Although this prior art measure may maintain hygiene of the insole of the footwear, it has a disadvantage such that the inside of an instep member or vamp cannot be kept clean even if the cover sheet is replaced. It is also not relevant to this application for the whole foot is involved here, and while it rests.

Another measure involves art in which an inner cover attached detachably to a part of a mule, at which the wearer's foot touches, the mule is replaced when soiled (Japanese Utility Model Registration No. 3008071(B)). There is no medication here.

This second measure may possibly overcome the shortcoming of the former measure noted above, but the work disadvantageously becomes onerous where the inner cover is replaced. Both are noncompetitive to this submission.

The following United States patents and/or other patent related documents were discovered during a search and appear to be non-similar, but possibly relevant to the functional and/or design features of this utility invention. These patent documents are not listed in any particular order, and a brief explanation of why each document was chosen is provided.

U.S. Pat. No. 6,430,845 discloses a sandal which has a detachable portion that is washable.

U.S. Pat. No. 6,684,442 discloses a disposable protective foot pad.

U.S. Pat. No. 3,903,620 discloses a clean sole attachment for shoes.

United States Patent Application 2007/0098981 discloses protective sheets for shoes.

U.S. Pat. No. 2,985,970 discloses a removable protective sheet for footwear, slipper.

United States Patent Application 2004/0261290 discloses a sandal sock.

U.S. Pat. No. 6,442,870 discloses a shoe with a replaceable vamp and insole.

United States Patent Application 2005/0241181 discloses a removable inner sole assembly for a sandal.

U.S. Pat. No. 7,222,440 discloses a resilient strap on sole cover.

U.S. Design Pat. Des. 374,549 illustrates a design for a sandal.

None of the prior art or patents comprise what the proposed invention achieves. First of all, all the prior art focuses on attachments or usage with the shoe or sandal. Such is not the case here. Secondly none of the prior art is medicated, and none can effectively be worn without the shoe or sandal. Indeed, there is no reason to use these patents or devices without the shoe or sandal. Here the device exists and has a utility above and apart from the shoe or sandal. The footwear becomes irrelevant. The device does protect the footwear, and does extends its life, but the utility here is far beyond usage as protection. The foot is being relaxed, medicated, healed, whether or not footwear is worn, and also can heat the foot during wearing. It has multiple usage.

### SUMMARY OF THE INVENTION

To attain the multiple objectives described above according to this invention, there is provided foot covering comprised by an elastic covering, medicated and heated for placement upon the foot or feet, to be worn when the foot is in motion or at relaxation.

The elastic device covers the foot, and upon usage, begins to heat and medicate the same. The device has no sole, no sole pad and is not attached to the sandal or shoe. It is not fastened to the shoe, is not fitted to the shoe, and is disposable after one usage.

The medication presently proposed for usage is similar to that used in heating pads for shoulders backs and thighs. It is long lasting, and works whether or not the body is in motion.

The objects or objectives of the invention are multiple:

1. Covering and heating the foot, whether or not in motion;

2. Medicating the foot for healing, whether or not in motion;

3. Protection of the shoe, or sandal, by the existence of the device, which will not break during usage, and will prevent soiling of the foot wear;

4. A relaxation device for the body at rest, as the device can be worn by a person sitting down, after work or exercise as a therapeutic;

5. The device is disposable and low cost, and biodegradable. A low cost shoe market or sandal market will not be invaded by the product.

### BRIEF DESCRIPTION OF THE DRAWINGS

Drawings will follow; a sample device will be submitted.

### CAVEAT

There are no figures with a shoe or sandal. The footwear is irrelevant to the device. The device can be worn under a sock, or be worn without a sock. It is elastic to the extent necessary to cover both roles.

### DESCRIPTION OF THE PREFERRED EMBODIMENTS

There are obvious nuances that can be applied to the device. The list is endless of the medications that can be used, or the healing devices in chemical form that could become part of the device. The opening device focuses on heat, healing, and relaxation. But specific medications for athelete's foot and other diseases and conditions of the feet will be explored.

What is claimed is:

1. A medicated footwear comprising:
an elastic foot covering attached to the foot directly to heat, medicate, insulate the foot, and as a side benefit, protect and sanitize the shoe or sandal.

2. A device specifically attached to the foot itself, unrelated to the shoe or sandal, that provides multiple benefits to the user, whether or not the shoe or sandal is being worn.

3. A disposable medicinal device for the foot that provides healing and heat, and can be worn at rest or while in motion.
